(19) United States
(12) Patent Application Publication
(10) Pub. No.: US 2007/0077551 A1
(43) Pub. Date: Apr. 5, 2007

(54) Sample analyzer

(75) Inventor: Hideki Hirayama, Akashi-shi (JP)

Correspondence Address: BRINKS HOFER GILSON & LIONE, P.O. BOX 10395, CHICAGO, IL, 60610, US

(73) Assignee: Sysmex Corporation

(21) Appl. No.: 11/542,518

(22) Filed: Oct. 3, 2006

(30) Foreign Application Priority Data
Oct. 3, 2005 (JP) 2005-290251

(51) Int. Cl. C12Q 1/00; G06F 19/00; C12M 3/00

(52) U.S. Cl. 435/4; 702/19; 435/287.2

(57) ABSTRACT

A sample analyzer for analyzing samples of a plurality of species of animals, comprising: a display; a memory for storing first icon data representing a first icon and second icon data representing a second icon, the first icon showing a first species of animal and being used for selecting a first analysis for a sample from the first species of an animal, and the second icon showing a second species of animal and being used for selecting a second analysis for a sample from the second species of an animal; a screen displayer for displaying a screen on the display, the screen comprising the first icon; and a change receiver for receiving a change of the first icon on the screen to the second icon is disclosed.

This application claims priority under 35 U.S.C. §119 to Japanese Patent Application No. 2005-290251 filed Oct. 3, 2005, the entire content of which is hereby incorporated by reference.

### FIELD OF THE INVENTION

The present invention relates to a sample analyzer for analyzing samples of a plurality of species of animals.

### BACKGROUND

Recently, veterinaries and livestock testing facilities have been conducting analyses of biological samples of animals such as blood and urine. Analyses of such biological samples are performed using analyzers such as those described in, for example, US Laid-Open Patent Publication Nos. 2005/0053521 and 2003/0070498. Since a wide variety of animal species are dealt with in these veterinaries and livestock testing facilities, these analyzers must be capable of performing analyses of a plurality of animal species. The analyzers disclosed in the above mentioned patent publications allow selection of an animal species, and are capable of analyzing biological samples under analysis conditions that conform to the selected animal species.

FIG. 22 shows an example of the display screen of an analyzer of the conventional type. As shown in the drawing, the screen 101 includes two species selecting icons 102 and 103, a selection button 104, and a start button 105. The species selection icon 102 is for selecting dog as the analysis object, and displays a picture of a dog. The species selection icon 103 is for selecting cat as the analysis object, and displays a picture of a cat. The selection button 104 is used to set other species as the analysis object. When the selection button 104 is selected, a dialogue is displayed for the selection of another species as the analysis object, and a list of icons that correspond to other species is displayed (not shown in the drawing). The selection button 104 is provided because it is difficult to display a plurality of icons arrayed on the main screen 103 since a large screen display unit can not be provided due to the need for the animal species analyzer to be compact for use in smaller veterinaries and the like. Therefore, the user sets the analysis object species using the species selection icons 102 and 103, and the selection button 104. Then, when the start button 105 is operated (clicked) after the analysis object species has been set, analysis is performed under the analysis conditions corresponding to the species set as the analysis object.

In the above mentioned conventional analyzers, however, the species selection icons 102 and 103 displayed on the screen 101 that receives the analysis start instruction can not be changed to the icon of another species, and when analyzing a species other than dog or cat, intricate manual operations are required to set the species of the analysis object using the selection button 104.

Due to the recent pet boom, ferrets and prairie dogs are among so-called exotic animals widely being raised as pets, and considerable numbers of these species are being cared for in veterinaries. Furthermore, cows and horses and such livestock species are more likely dealt with than dogs and cats in livestock testing facilities. Therefore, the species being dealt with certainly differ depending on whether the facility is an urban veterinary or rural livestock testing facility, such that it becomes very inconvenient when it is necessary to set the species of the analysis object through complex labor for each analysis at those facilities that cater to species other than dogs and cats.

### SUMMARY

The scope of the present invention is defined solely by the appended claims, and is not affected to any degree by the statements within this summary.

A first aspect of the present invention is a sample analyzer for analyzing samples of a plurality of species of animals, comprising: a display; a memory for storing first icon data representing a first icon and second icon data representing a second icon, the first icon showing a first species of animal and being used for selecting a first analysis for a sample from the first species of an animal, and the second icon showing a second species of animal and being used for selecting a second analysis for a sample from the second species of an animal; a screen display means for displaying a screen on the display, the screen comprising the first icon; and a change receiving means for receiving a change of the first icon on the screen to the second icon.

A second aspect of the present invention is a sample analyzer for analyzing samples of a plurality of species of animals, comprising: a display; a memory for storing first icon data representing a first icon and second icon data representing a second icon; the first icon showing a first species of animal and being used for selecting a first analysis of a sample from the first species of an animal, and the second icon showing a second species of animal and being used for selecting an analysis of a sample from the second species of an animal; a screen display means for displaying a screen on the display, the screen comprising the first icon on a first position of the screen and the second icon on a second position of the screen; and a change receiving means for receiving a change of a position of the first icon from the first position to the second position and a change of a position of the second icon from the second position to the first position.

A third aspect of the present invention is a sample analyzer for analyzing samples of a plurality of species of animals, comprising: a display; a memory for storing first icon data representing a first icon and second icon data representing a second icon; the first icon showing a first species of animal and being used for selecting a first analysis of a sample from the first species of an animal, and the second icon showing a second species of animal and being used for selecting a second analysis of a sample from the second species of an animal; a screen display means for displaying a screen on the display, the screen comprising at least one first icon; and a change receiving means for receiving a change of a number of species of animals shown on the screen.

### BRIEF DESCRIPTION OF THE DRAWINGS

FIG. 1 is a perspective view briefly showing the structure of a biological sample analyzer of an embodiment of the present invention;

FIG. 2 shows the structure of the control unit;

FIG. 3 is a flow chart showing the operation flow of the biological sample analyzer of the embodiment of the present invention;

FIG. 4 shows a main screen;

FIG. 5 shows a screen that displays the measurement object species selection dialogue;

FIG. 6 shows screen during measurements;

FIG. 7 is a graph showing an example of the particle size distribution of white blood cells in one species of animal;

FIG. 8 is a graph showing an example of the particle size distribution of white blood cells in another species of animal;

FIG. 9 is a flow chart showing the flow of the operation to change the species selection icon of the biological sample analyzer of the embodiment of the present invention;

FIG. 10 shows a menu screen;

FIG. 11 shows an example of a first species selection screen;

FIG. 12 shows a second species selection screen;

FIG. 13 shows a species name setting screen;

FIG. 14 shows a first abnormality setting screen;

FIG. 15 shows a correction value setting screen;

FIG. 16 shows a first fraction position setting screen;

FIG. 17 shows another example of a first species selection screen;

FIG. 18 shows an example of an icon allocating screen;

FIG. 19 shows a screen displaying a species selection dialogue;

FIG. 20 shows another example of an icon allocating screen;

FIG. 21 shows the main screen after the icon allocation change; and

FIG. 22 shows an example of the display screen of an analyzer of the conventional type.

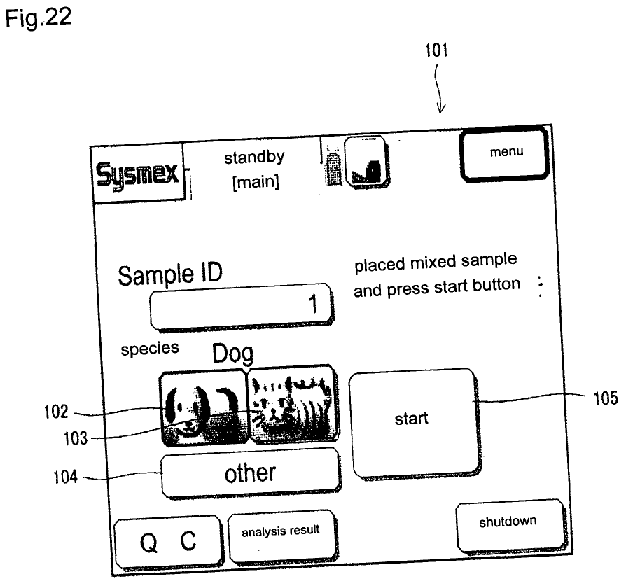

### DETAILED DESCRIPTION OF THE PREFERRED EMBODIMENTS

The preferred embodiments of the present invention are described hereinafter with reference to the drawings.

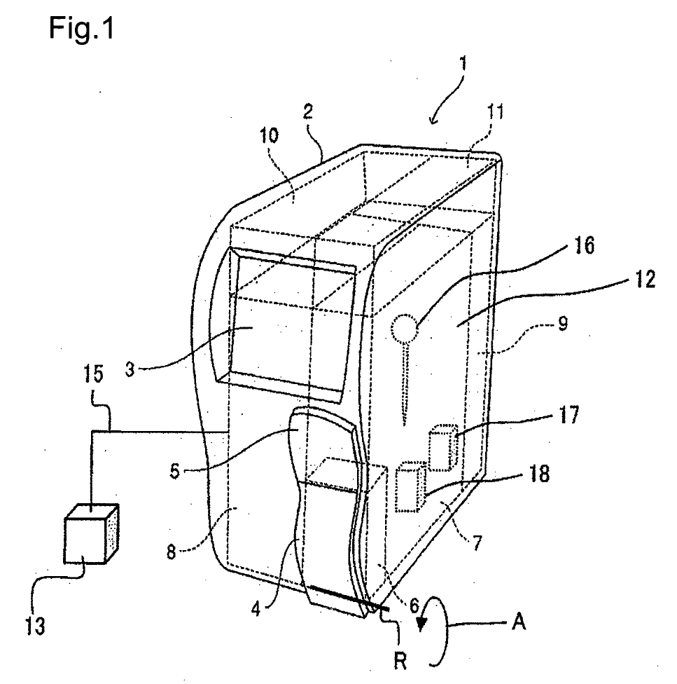

FIG. 1 is a perspective view briefly showing the structure of a biological sample analyzer of an embodiment of the present invention. The biological sample analyzer 1 is a blood cell counter that counts red blood cells and white blood cells and the like of animal species such as dog, cat, cow, horse and the like. The biological sample analyzer 1 is capable of operating in operation modes that correspond to each species of animal, such as a dog analysis mode for analyzing the blood of dogs, cat analysis mode for analyzing the blood of cats and the like.

As shown in FIG. 1, the biological sample analyzer 1 is provided with a housing 2, display 3, sample setting panel 4, button 5, controller 9, power source 10, printer 11, and measuring mechanism 12. The housing 2 is an approximately rectangular shaped box, which internally accommodates the display 3, controller 9, power source 10, printer 11, and measuring mechanism 12. The display 3 is a touch panel type liquid crystal display that is mounted on the upper front surface of the housing 2. Information can be display and user input can be received via the display 3. Below the display 3 of the housing 2 is hollowed, and this hollow part is closed by a door-like sample setting panel 4. The bottom end of the sample setting panel 4 pivots on an axle R that extends in the lateral direction of the housing 2, and the sample setting panel 4 is rotatable in a forward direction (arrow A direction in the drawing) via the axle R. The button 5 is disposed above the sample setting panel 4, and the sample setting panel 4 and the button 5 are mutually connected. When a user presses the button 5, the connection of the sample setting panel 4 and the button 5 is released, and the sample setting panel 4 rotates upward. Furthermore, when the sample setting panel 4 is in an opened condition and the user presses the panel 4 backward, the sample setting panel 4 and the button 5 are reconnected via the rotation, such that the sample setting panel 4 maintains a closed condition.

A sample setting unit 6 is provided at the back part of the sample setting panel 4. The sample setting unit 6 has an open top to allow a sample container that contains a sample (biological sample) to be placed within the unit through the open top. The sample setting unit 6 rotates integratedly with the sample setting panel 4, such that a user can place a sample container in the sample setting unit 6. A user who has placed a sample container presses the sample setting panel 4 backward so as to return the sample setting unit 6 to the closed position shown in FIG. 1. Thus, a sample can be measured.

The measuring mechanism 12 is provided with a sample processing part 7 and a fluid controller 8. The sample processing part 7 is provided with a suction mechanism 16, detecting part 17, and mixing chamber 18. The suction mechanism 16 suctions the sample from the sample container placed in the sample setting part 6 and injects the sample into the detecting part 17 and mixing chamber 18; the mechanism is provided with a suction tube and a motor or the like for moving the suction tube. The fluid controller 8 is connected to a reagent container 13 that contains reagent via a tube 15, and is provided with a pump for delivering the reagent, and a motor or the like for actuating the pump. The sample and reagent are injected into the detecting part 17 and mixing part 18, and a discharged therefrom, via the fluid controller 8. The mixing chamber 18 is used to adjust the measurement sample by mixing the sample and reagent, and the sample that has been adjusted by the mixing chamber 18 is supplied to the detecting part 17. The detecting part 17 detects blood cells by a well known electrical resistance detection method, and outputs electrical signals that represent the characteristics pf the blood cells in the sample. Furthermore, the detecting part 17 detects the opacity (light absorption) of the sample by a well known optical detection method, and outputs electrical signals representing this opacity. Specifically, the detecting part 17 outputs electrical signals representing white blood cells, red blood cells, hemoglobin, and platelets contained in the sample. These output signals are sent to the controller 9, which processes these signals to obtain data representing the size (volume) of each detected particle (blood cell), and data representing the opacity (light absorption) of the sample as measurement data. The controller 9 controls the operation of each of these units and calculates the analysis results. The power supply 10 converts the alternating current (AC) from a commercial AC power source to a direct current (DC), and supplies this direct current to the controller 9 and motors and the like of each unit. The printer 11 receives instructions from the controller 9 and prints the analysis results and the like.

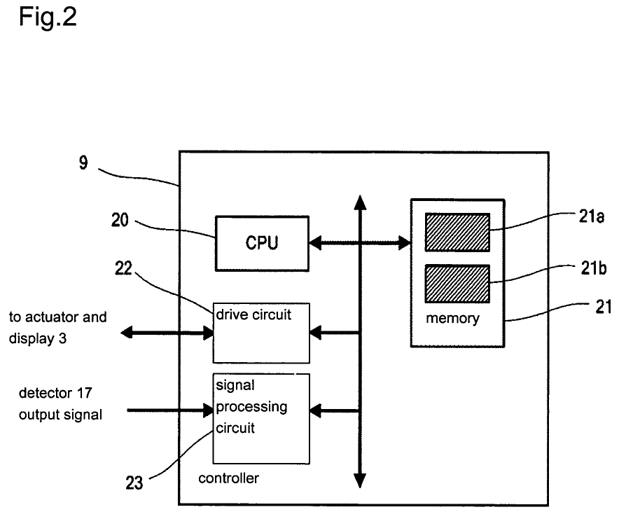

The structure of the controller 9 is described in detail below. FIG. 2 shows the structure of the controller 9. The controller 9 is provided with a CPU 20, memory 21, drive circuit 22, and signal processing circuit 23. The CPU 20 is capable of executing programs stored in the memory 21, and the biological sample analyzer 1 is operated in a manner described later when the CPU 20 executed these programs. The memory 21 is configured, for example, by RAM such as SRAM, DRAM or the like, ROM such as a mask ROM, EEPROM or the like, flash memory (registered trademark) or the like; the memory 21 contains pre-stored programs, stores data generated during program execution, and stores measurement data and analysis results and the like. The memory 21 contains two areas: area 21a and area 21b.

The area 21a stores an application program for overall control of the biological sample analyzer 1, a measurement control program for controlling the measurement operation and controlling the operation of the motors and the like of the fluid controller 8 and suction mechanism 16, and analysis program for obtaining analysis results by analyzing measurement data obtained by signal processing the electrical signals output from the detection part 17. The area 21b is used jointly by these programs. The area 21b stores analysis result data D which represent the analysis results obtained by the biological sample analyzer 1. The analysis result data are obtained for each sample and stored in the area 21b for a predetermined number of samples (for example, twenty samples) sequentially from the newest. The analysis result data D include measurement data D1 obtained by signal processing the electrical signals detected by the detecting part 17, and analysis result data D2 representing the analysis results of the analysis program.

The drive circuit 22 receives the instructions from the measuring operation program, and controls the actuation of the motors, actuators such as a electromagnetic valves (not shown in the drawing, and display 3. The signal processing circuit 23 performs predetermined signal processing, for example, noise elimination process, A/D conversion processing, high pulse detection processing and the like on the electrical signals obtained by the detecting part 17, and outputs the measurement data D1 as digital data. The obtained measurement data D1 are intermediate data representing the properties of the sample until arriving at the analysis data D2, and are stored in the area 21b of the memory 21.

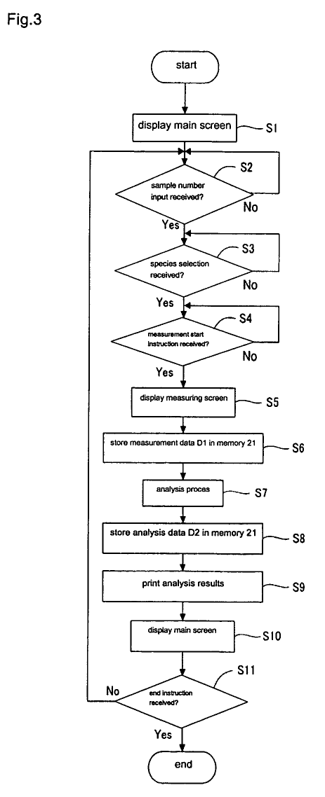

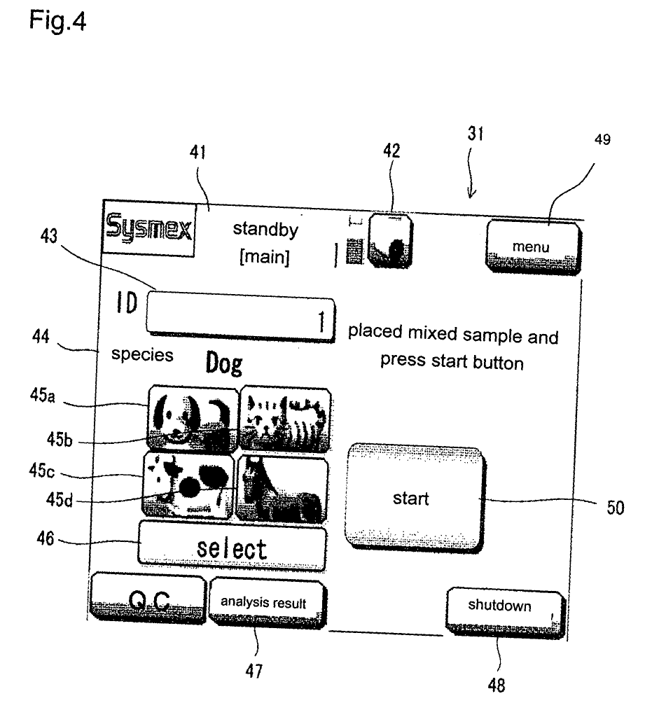

The operation of the biological sample analyzer 1 of the embodiment of the present invention is described below. FIG. 3 is a flow chart showing the operation flow of the biological sample analyzer 1 of the embodiment of the present invention. First, the user starts the biological sample analyzer 1. Immediately after start-up, the controller 9 displays the main screen on the display 3 (step S1). FIG. 4 shows a main screen. The main screen 31 is provided with an analyzer status display area 41, paper feed button 42, sample number display area 43, animal species display area 44, animal species selection icons 45a through 45d, selector button 46, analysis result button 47, shutdown button 48, menu screen call button 49, start button 50 and the like. At this time, the message “standby” is displayed on the analyzer status display area 41 since a sample was not first placed in the biological sample analyzer 1. This “standby” display indicates that the measurement can start. In this condition in which the main screen is displayed, a user presses the button 5, the sample setting panel 4 rotates forward, and a sample is placed in the sample setting unit 6. Then, the user rotates the sample setting panel 4 backward until the sample setting panel 4 engages the button 5 such that the sample is set in the biological sample analyzer 1.

Next, the controller 9 receives the sample number input by the user (step S2: YES). The sample number input is accomplished as follows. When the user touches the sample number display area 1 of the main screen 31 with a fingertip, a software keyboard is displayed on the display 3. The user can input the sample number by touching the software keys with a fingertip. Then, the controller 9 selects the measurement animal species, that is, receives the setting for the operating mode (step S3: YES). The animal species selection icon 45a on the main screen 31 is assigned to dog, 45b is assigned to cat, 45c is assigned to cow, 45d is assigned to horse, and the assigned animal species is illustrated in each icon. Although a picture of the animal species is shown in the icons in FIG. 4, the illustration is not limited to this mode inasmuch as, for example, text may be displayed to indicate the animal species. If the animal species object is shown in one of the four icons, the user touches that animal species icon with a fingertip. Thus, that animal species is set as the measurement object (measurement animal species). At this time, the icon of the set measurement animal species is displayed in a different way than the other icons, such as only the icon of the set as the measurement animal species is displayed in color and the other icons are displayed in black and white so as to allow the user to easily confirm which species has been set as the measurement species.

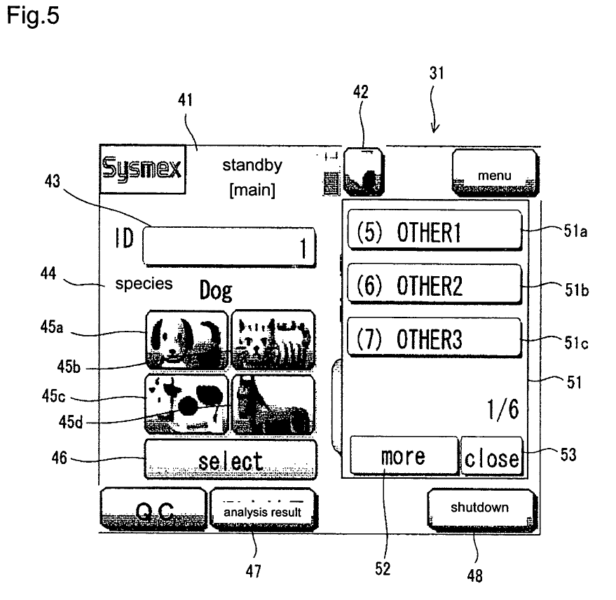

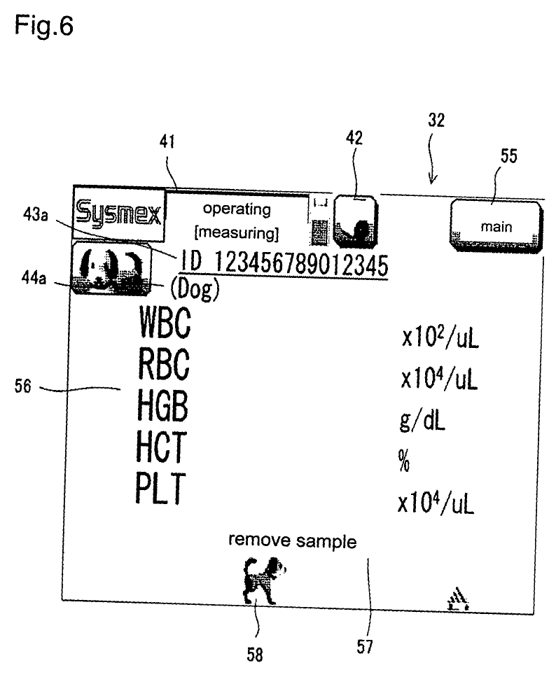

Furthermore, the user performs the following operation to select an animal species that is not represented by one of the four icons as a measurement animal species. First, the user touches the selector button 46 with a fingertip. When the selector button 46 is selected in this way, a measurement species selection dialogue is displayed. FIG. 6 shows a screen that displays the measurement species selection dialogue. A plurality of buttons 51a through 51c used for animal species selection are aligned and displayed in the measurement species selection dialogue 51. A page switching button 52 and close button 53 are also displayed in the measurement species selection dialogue 51. The buttons 51a through 51c can be allocated to optional animal species by the user, and the name of the allocated animal species can be displayed. In the example shown in FIG. 5, the user has not made any allocations, and the buttons 51a through 51c are displayed as “OTHER 1,” “OTHER 2,” and “OTHER 3.” When one of these buttons 51a through 51c is selected, the controller 9 sets the animal species allocated to the selected button as the measurement species. That is, the desired species of animal is set as the measurement species when the user touches the button of the desired species with a fingertip in the dialogue 51. The measurement species selection dialogue 51 is configured by a plurality of pages, such that when the page switching button 52 is selected, the controller 9 switches the display to another page. Therefore, when the button of a desired species is not displayed on the page, the user touches the page switching button 52 with a fingertip, and the display is switched to another page. Furthermore, when the close button 53 is selected, the dialogue 51 is closed.

The controller 9 receives the instruction to start measurement (step S4: YES). When the user specifies the start of measurement by touching the start button 50 with a fingertip. When the start button 50 is selected in this way, the controller 9 starts the measurement operation and displays the measuring screen on the display 3 (step S5).

FIG. 6 shows the measuring screen. As shown in FIG. 6, the measuring screen 32 is, similar to the main screen 31, provided with an analyzer status display area 41, paper feed button 42, sample number display area 43, and species display area 44, as well as a main screen call button 55, analysis result display area 56, message display area 57, and measurement progress status display area 58. The measuring screen 32 displays a line of text saying “Performing operation” in the analyzer status display area 41. When the user selects the main screen call button 55 in the measuring screen 32, the display of the display 3 is switched to the main screen 31. After the sample needed for measurement has been suctioned from the sample container, the message “Removing sample” is displayed in the message display area 57 as shown in the drawing. At this time, the user can open the sample panel 4 and remove the sample. The measurement operation progress status display area 58 is a band at the bottom edge of the screen, and the time needed for the measurement may be associated along the entire lateral length. That is, the left end can indicate the measurement starting time and the right end can indicate the measurement ending time, and the image of a dog can move from the left end to the right end in conjunction with the progress of the measurement operation. FIG. 6 shows the measuring screen in the dog analysis mode, and the display changes according to the species of animal being measured; that is, the image of a cat is shown to indicate the progress position in the cat analysis mode and the like.

When the measurement start in this way, blood cells in the sample are measured by the detecting part 17, and the measurement data D1 are stored in area 21b of the memory 21. Then, the controller 9 executes the sample analysis process using the measurement data D1 (step S7). This analysis process is executed under the analysis conditions according to the species of animal set in step S3.

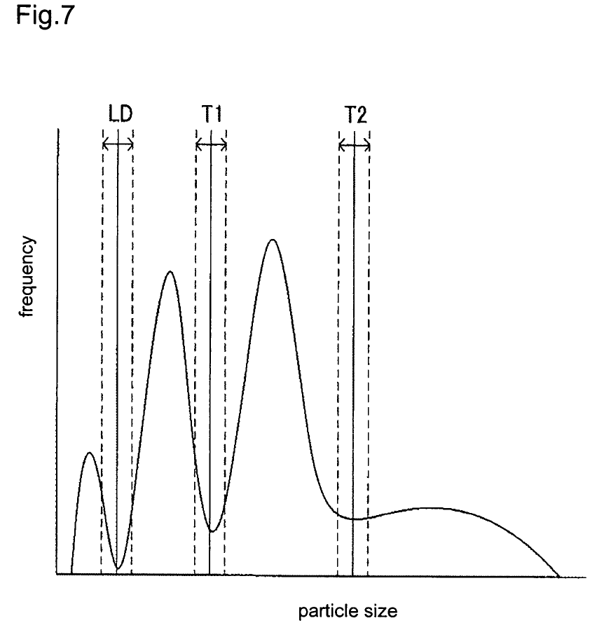

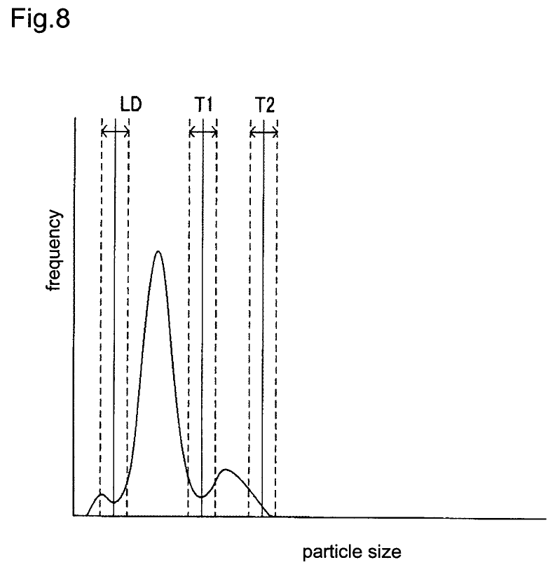

The analysis of the sample by the biological sample analyzer 1 is described in detail below. A sample prepared by mixing a hemolytic agent with the blood sample is used in the measurement of white blood cells in the measuring mechanism 12. The electrical signal output from the detecting part 17 is a pulse signal that represents the size of the particle, and the measurement data D1 representing the size of the particle are obtained by signal processing the electrical signal. Therefore, it is possible to create a particle size distribution using the measurement data D1. FIG. 7 is a graph showing an example of a size distribution of white blood cells in one animal species (for example, dog), and FIG. 8 is a graph showing an example of a size distribution of white blood cells in another animal species (for example, rabbit). As shown in the drawings, the size distribution of the white blood cells differ markedly between the two different species of animals. This difference is due to the difference in the size of the white blood cells differs by species. When measuring white blood cells, the red blood cells are destroyed by adding hemolytic agent to the sample to eliminate the influence of red blood cells before measurement. The cell membrane of the destroyed red blood cells remain in the sample after the addition of the hemolytic agent, and these are measured together with the white blood cells. Furthermore, lymphocytes, monocyte, eosinophils, basophils, neutrophils and the like are present among the white blood cells, and these types have respectively difference particle sizes depending on the type. Therefore, in the analysis of white blood cells performed by the biological sample analyzer 1, the blood cells are classified (fractioned), for example, as large white cells corresponding to neutrophils, monocytes, eosinophils, medium white cells corresponding to basophils, small white cells corresponding to lymphocytes, and red blood cell ghosts (cell membrane of the residual red blood cells). In the examples shown in FIGS. 7 and 8, white blood cells and red blood cell ghosts are fractioned by a first fraction level LD, small white cells and medium white cells are fractioned by second fraction level T1, and medium white cells and large white cells are fractioned by a third fraction level T2. These fraction levels differ depending on the species, and accurate analysis can not be performed unless the fraction levels are appropriately set according to the species of animal being measured. More specifically, since the fraction levels differ depending on the sample even in the same species of animal, a range is set for the fraction level for each species of animal (indicated by the dashed line), and the trough position of the particle size distribution is searched within this range and the position of the trough obtained by this search is set as the fraction level, as shown in FIGS. 7 and 8. In this case, an accurate fraction level can not be obtained unless the search range of the fraction level is set according to the species of animal. For this reason, the analysis conditions are preset for each species of animal in the biological sample analyzer 1, and when the measurement species is set, the analysis is executed pursuant with the analysis conditions for that animal species.

When the analysis process ends, the controller 9 stores the obtained analysis data D2 in area 21b of the memory 21 (step S8), the analysis results are printed on paper by the printer 11 (step S9), the main screen 31 is again displayed on the display 3 (step S10), and the next measurement is awaited. Since the analysis results are printed on paper by the printer 11, the user can easily confirm the analysis results.

When a user wants to continue the analysis (step S11: NO), the next sample is placed in the biological sample analyzer 1, and measurement is specified. Thus, it is possible to efficiently analyze a plurality of samples continuously without the user performing an operation to display the main screen 31 when starting the analysis of the next sample by switching the display automatically to the main screen 31.

When the user wants to end the analysis of a sample in the biological sample analyzer 1, the user touches the shutdown button 48 on the main screen 31 using a fingertip. When the selection of the shutdown button 48 is received in this way (step S11: YES), the controller 9 stops the biological sample analyzer 1.

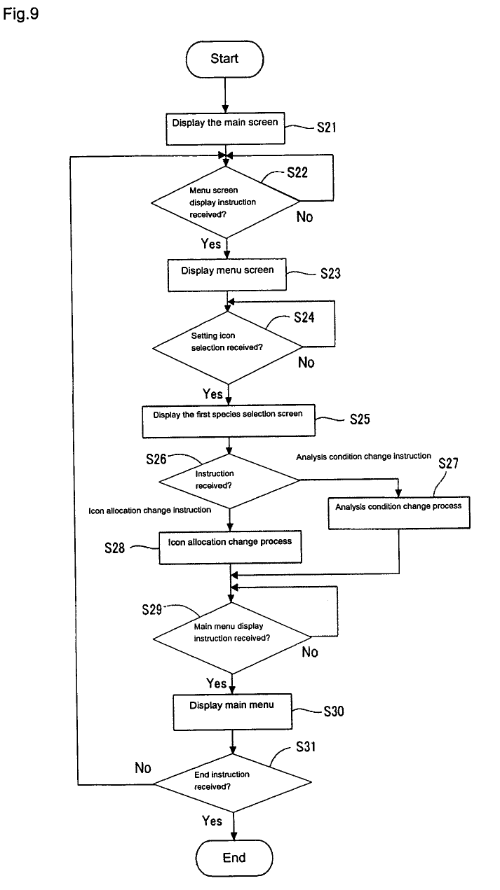

The operation for changing the species selection icon is described below for the biological sample analyzer 1 of the embodiment of the present invention. When changing the species selection icons 45a through 45d of the main screen 31, the biological sample analyzer 1 is operated as described below. The following description pertains to allocating rabbit to the species selection icon 45d. FIG. 9 is a flow chart showing the flow of the operation to change the species selection icon of the biological sample analyzer 1 of an embodiment of the present invention. The user starts the biological sample analyzer 1, and the controller 9 displays a main screen on the display 3 (step S21). The fingertip of the user touches the menu screen call button 49 on the main screen 31 to specify the display of the menu screen. When the menu screen display instruction is received (step S22: YES), the controller 9 displays the menu screen on the display 3 (step S23).

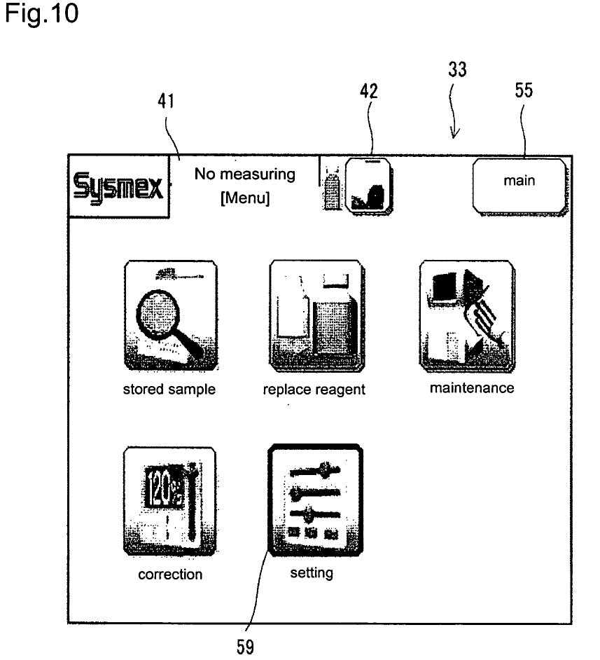

FIG. 10 shows the menu screen. As shown in the drawing, the menu screen 33 is, similar to the main screen 31, provided with an analyzer status display area 41 and paper feed button 42, as well as a main screen call button 55 and set icon 59. A line of text saying “Measurement prevented” is displayed in the analyzer status display area 41 of the analysis result screen 33, indicating that measurement can not start. The user selects the main screen call button 55 in the menu screen 33 to discontinue the changing of the setting of the species selection icon. However, when the user continues to change the setting of the species selection icon, the fingertip of the user touches the setting icon 59 in the menu screen 33 specify the display of a screen for changing the setting of each species. When the selection of the setting icon button 59 has been received (step S24: YES), the controller 9 displays a first species selection screen on the display 3 (step S25).

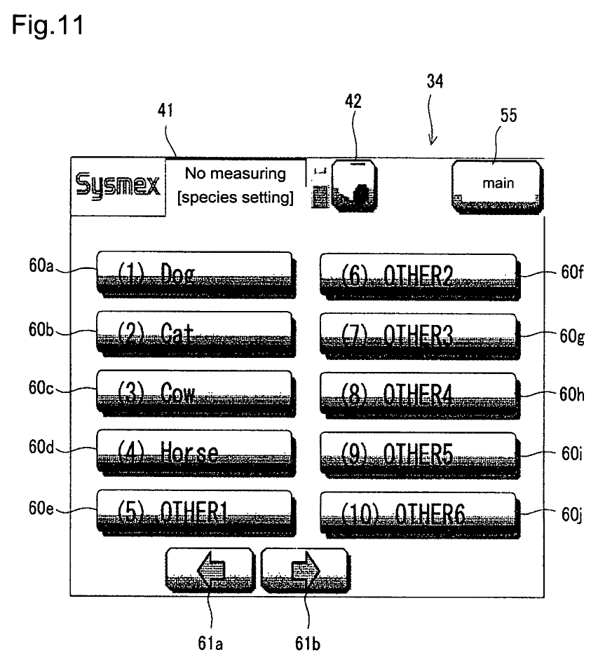

FIG. 11 shows an example of a first screen for species selection. This screen is displayed by selecting the setting button 59 displayed in the menu screen 33 shown in FIG. 10. As shown in FIG. 11, the first animal species selection screen 34 is provided with an analyzer status display area 41, paper feed button 42, and main screen call button 55, and also provided with ten species buttons 60a through 60j that respectively correspond to various species, and change screen buttons 61a and 61b. The species button 60a is allocated for dog and displays “Dog.” Similarly, the species buttons 60b, 60c, and 60d are respectively allocated for cat, cow, and horse, and respectively display “Cat,” “Cow,” and “Horse.” The species buttons 60e through 60j are user-allocatable for optional species, and display the name of the allocated species. In the example shown in FIG. 11, the user has not allocated these buttons, such that the buttons 60e through 60j display “Other 1,” “Other 2,” and “Other 3.” When a user touches the change screen buttons 61a and 61b with a fingertip in the first species selection screen 34, a second species selection screen is displayed.

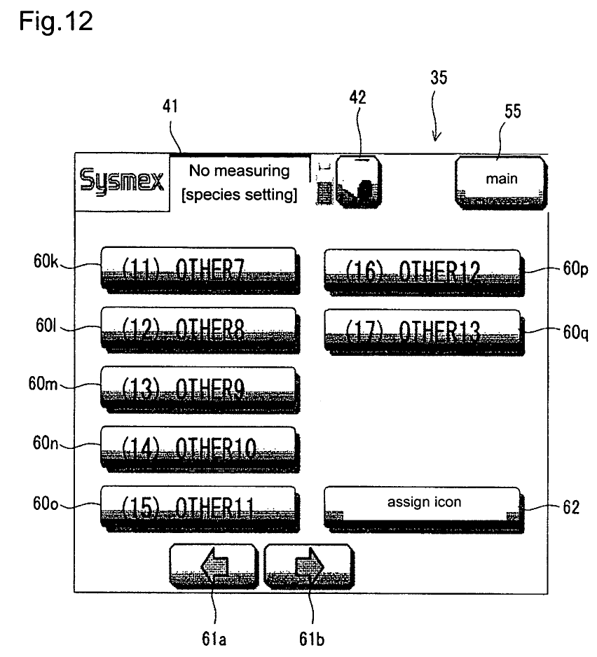

FIG. 12 shows a second species selection screen. As shown in the drawing, the second species selection screen 35 is provided with an analyzer status display area 41, paper feed button 42, main screen call button 55, and change screen buttons 61a and 61b, as well as seven species buttons 60k through 60q, and an icon allocation button 62. The species buttons 60k through 60q are user-allocatable for optional species, and display the name of the allocated species. In the example shown in FIG. 12, the user has not allocated these buttons, such that the buttons 60k through 60q display “Other 7,” “Other 8,” and “Other 9.” Furthermore, when the change screen buttons 61a and 61b are selected, the display is switched to the previously described first species selection screen 34.

When changing the setting of the analysis condition of a species, the user selects the species button among buttons 60a through 60q corresponding to the species of the desired setting change in the first species selection screen 34 and the second species selection screen 35, and the icon allocation button 62 is selected when changing the setting of the species selection icons 45a through 45d. When the analysis condition setting change instruction is received from the user, that is, when a species button 60a through 60q has been selected (“Analysis condition setting change instruction” in step S26), and when the analysis condition setting change process (step S27) is executed and the setting change instruction of a species selection icon 45a through 45d has been received, that is, when the selection of the icon allocation button 62 has been received (“Icon allocation change instruction” in step S6), the icon allocation change process (step S28) is executed.

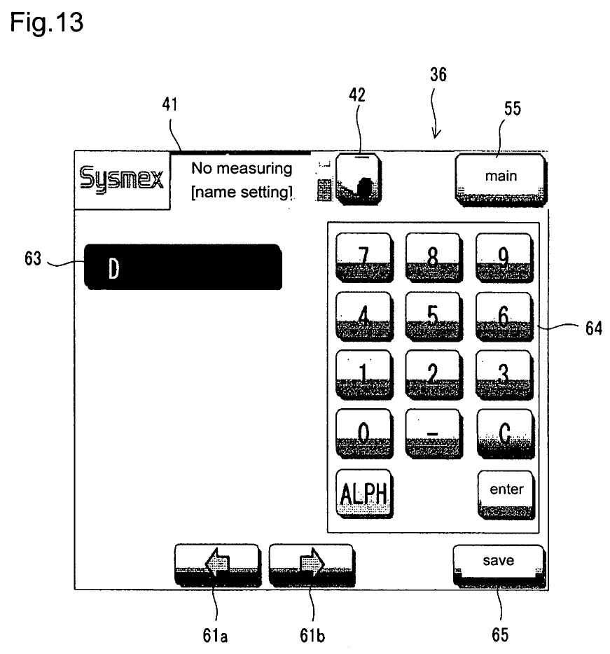

The analysis condition changing process is described in detail below. When changing the setting of the analysis conditions for dog, cat, cow, and horse, the user touches one of the animal species buttons 60a, 60b, 60c, or 60d with a fingertip. Thus, a first abnormality setting screen for the species corresponding to the selected button is displayed. The first abnormality setting screen is described later. When setting the analysis condition of another species, the user touches any one button among the species buttons 160e through 60q with a fingertip. Thus, the species name setting screen corresponding to the selected button is displayed on the display 3. FIG. 13 shows a species name setting screen. As shown in the drawing, the species name setting screen 36 is, similar to the first species selection screen, provided with an analyzer status display area 41, paper feed button 42, main screen call button 55, and change screen buttons 61a and 61b, as well as a text input display area 63, software keyboard 64, and save button 65. A user can input text (species name) using the software keyboard 64 in the species name setting screen 36. For example, when setting the analysis conditions for rabbit, the user inputs “Rabbit.” The text entered in this way is displayed in the text input display area 63. When the input content is saved, the user touches the save button 65 with a fingertip. Thus, the controller 9 associates the input text with the species button and stores the input text in the memory 21. The analysis condition setting screen is also provided with a first abnormality setting screen, second abnormality setting screen, third abnormality setting screen, fourth abnormality setting screen, fifth abnormality setting screen, correction value setting screen, first, fraction position setting screen, and second fraction position setting screen. As shown in the drawing, a left pointing arrow is displayed in the change screen button 61a, and a right pointing arrow is displayed in the change screen button 61b. The display on the display 3 is charged to another analysis condition setting screen whenever the user touches the change screen button 61a and 61b with a fingertip. The user presses selects the change screen buttons 61a and 61b until the desired analysis condition setting screen is displayed.

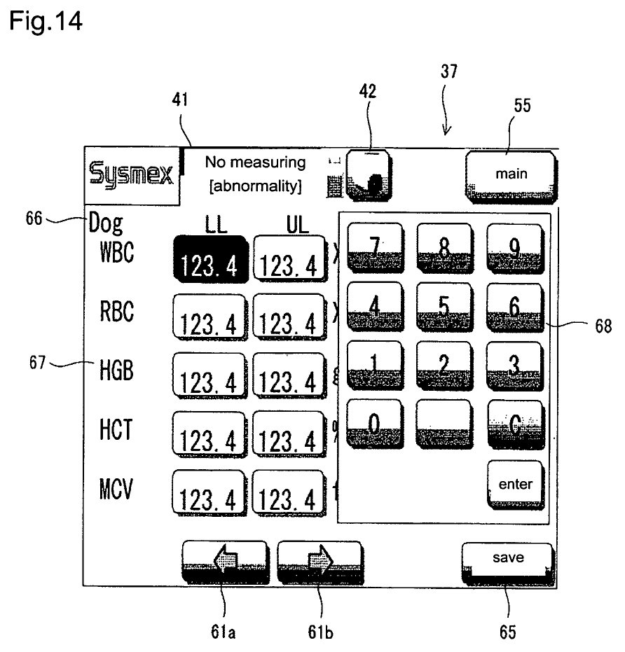

FIG. 14 shows a first screen for determining an abnormality. As shown in the drawing, the first abnormality setting screen 37 is, similar to the species name setting screen 36, provided with an analyzer status display area 41, paper feed button 42, main screen call button 55, and change screen buttons 61a and 61b, as well as a species display area 66, and abnormality value display area 67. In the first abnormality setting screen 37, it is possible to set an abnormality for white blood cell concentration (WBC), red blood cell concentration (RBC), hemoglobin concentration (HGB), hematocrit value (HCT), and mean red blood cell volume (MCV). Abnormalities can be set for mean corpuscular cell hemoglobin (MCH), mean corpuscular hemoglobin concentration (MCHC), and platelet concentration (PLT) in the second abnormality screen, abnormalities can be set for small white cell ratio (W-SCR), medium white cell ratio (W-MCR), and large white cell ratio (W-LCR) in the third abnormality setting screen, abnormalities can be set for small white cell concentration (W-SCC), medium white cell concentration (W-MCC), and large white cell concentration (W-LCC) in the fourth abnormality setting screen, and abnormalities can be set for red cell distribution range SD (RDW-SD), red cell distribution range CV (RDW-CV), platelet distribution range (PDW), mean platelet volume (MPV), and large platelet ratio (P-LCR) in the fifth abnormality setting screen. Setting the abnormality is accomplished by setting an upper limit value and lower limit value for a normal analysis value. That is, when an analysis value does not lie between the upper limit value and the lower limit value, the analysis value is deemed abnormal and a flag indicating an abnormality is displayed. The user selects an area by touching the area displaying a set value the user wants to change with a fingertip. When a set value area is selected, the software keyboard 68 is displayed, and the user can enter a new set value via the software keyboard 68. The input set value is displayed in the selected area to indicate the set value has been changed. The software keyboard 68 is not provided with an alphabetic software keys so that only numerals can be entered. After the abnormality setting has been changed in this way, the user saves the setting value by selecting the save button 65. Thus, the controller 9 stores the set value as an abnormality setting of the object animal species in the memory 21.

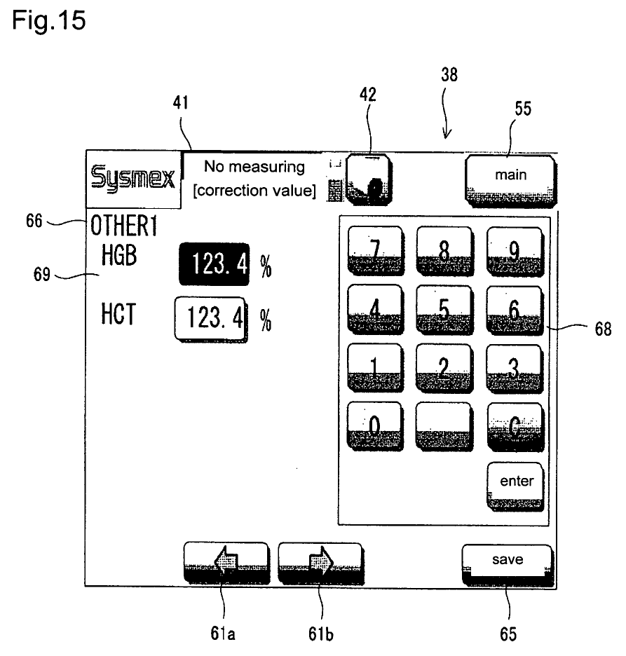

FIG. 15 shows a correction value setting screen. As shown in the drawing, the correction value setting screen 38 is, similar to the abnormality setting screen 37, provided with an analyzer status display area 41, paper feed button 42, main screen call button 55, change screen buttons 61a and 61b, save button 65, and species display area 66, as well as a correction value display area 69. The correction value can be set for hemoglobin concentration (HGB), and hematocrit value (HCT) in the correction value setting screen 38. The area is selected when the user touches the area displaying the correction value the user wants to change with a fingertip. When a correction value area is selected, a software keyboard 69 is displayed, and the user can input a new correction value via the software keyboard 69. The input correction value is displayed in the selected area to indicate that the correction value has been changed. After the correction value setting has been changed in this way, the user stores the correction value by selecting the save button 65. Thus, the controller 9 stores the correction value as the correction value of the object animal species in the memory 21.

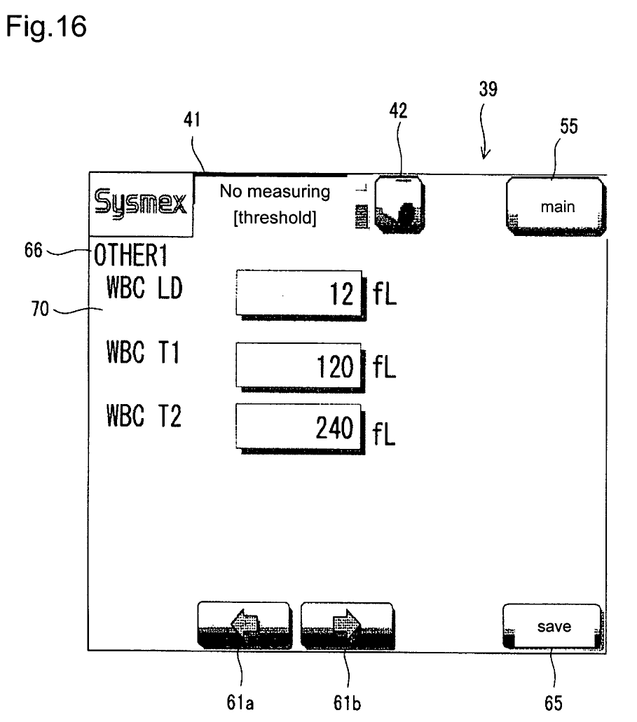

FIG. 16 shows a first screen for setting the fraction position. As shown in the drawing, the first fraction position setting screen 39 is, similar to the first abnormality setting screen 37, provided with an analyzer status display area 41, paper feed button 42, main screen call button 55, change screen buttons 61a and 61b, save button 65, and species display area 66, as well as a fraction level value display area 70. It is possible to set the white cell fraction levels LD, T1, and T2 in the first fraction position setting screen 138. Furthermore, the red cell fraction level and platelet fraction level can be set in the second fraction position setting screen. The user selects an area by touching the area displaying the setting value the user wants to change with a fingertip. When the setting value area is selected, a software keyboard (not shown in the drawing) is displayed, and the user can enter a new setting value via the software keyboard. The input setting value is displayed in the selected area to indicate the setting value has been changed. After the fraction level setting value has been changed in this way, the user stores the setting value by selecting the save button 165. Thus, the controller 9 stores the setting value as the fraction level of the object species in the memory 21. Among the species for which fraction level setting values have been stored, the analysis processing is performed using the fraction level. Among the four species of dog, cat, cow, and horse (or three species of dog, cat, and cow), the trough position of the particle size distribution is searched from a predetermined range, and this trough position is used as the fraction level.

Thus, after the settings of the analysis condition have been changed, the user calls the main screen by touching the main screen call button 55 with a fingertip. When the main screen 31 is called (step S29: YES), the controller 9 displays the main screen 31 on the display 3 (step S30). The user touches the shutdown button 48 on the main screen 31 with a fingertip to stop the operation of the biological sample analyzer 1. The biological sample analyzer 1 stops when the controller 9 receives the selection of the shutdown button 48 (step S31: YES). Furthermore, when the user wants to change another setting, the process returns to step S22, and the processes subsequent to step S22 are repeated.

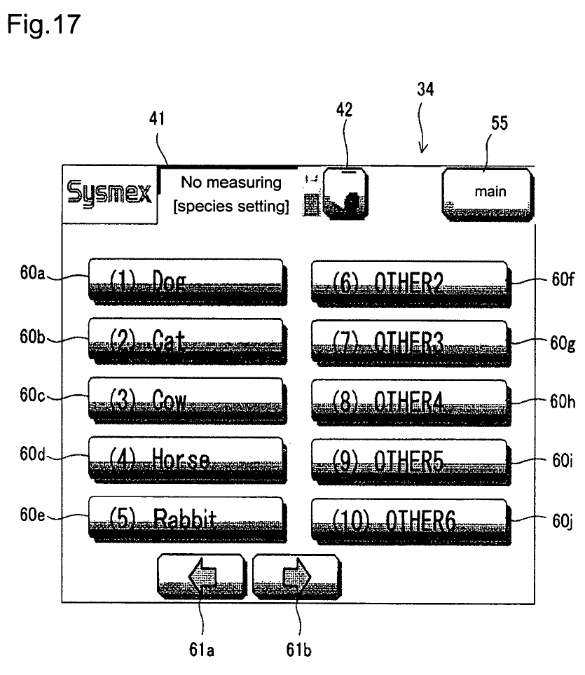

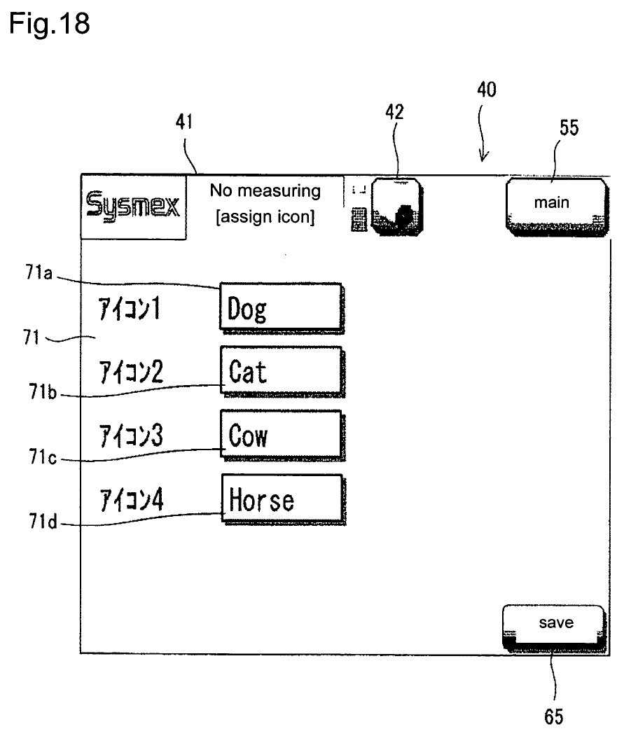

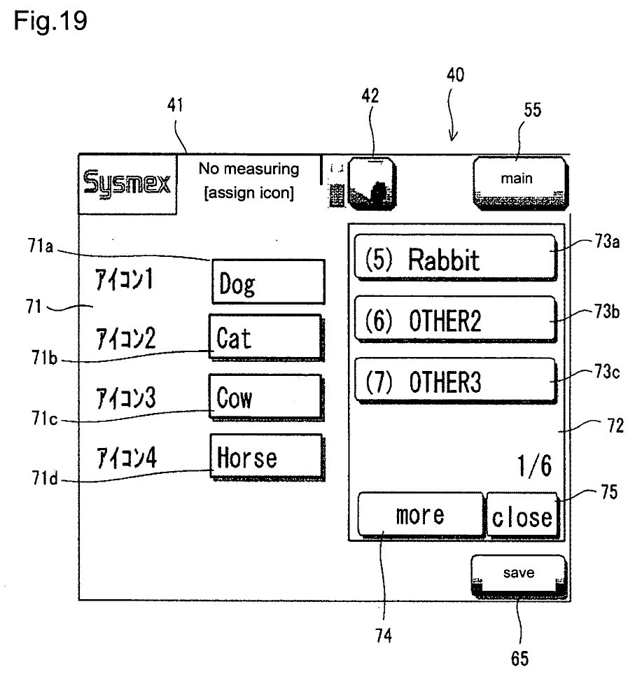

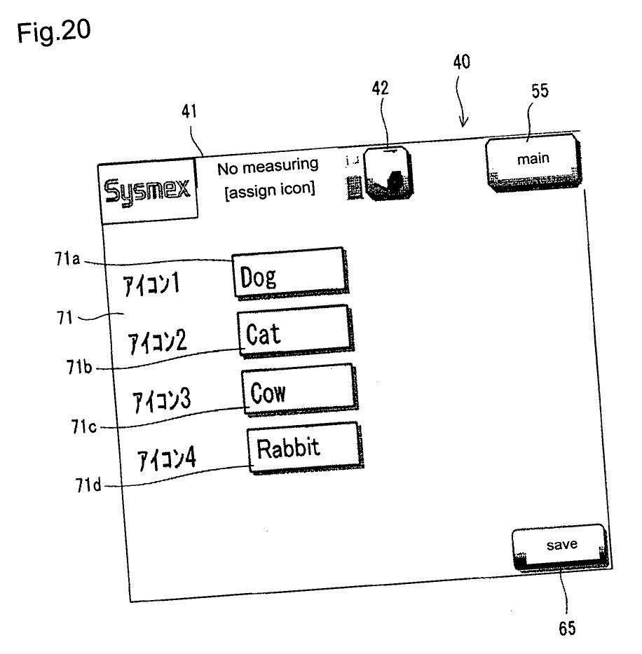

The icon allocation changing process is described in detail below. The following description pertains to the case in which rabbit is allocated to the species button 60e of the first species selection screen by means of the analysis condition setting change process described previously, such that rabbit is set as the analysis condition. FIG. 17 shows another example of a first species selection screen. In the example shown in the drawing, rabbit is allocated to the species button 60e, and “Rabbit” is displayed. That is, rabbit is set as the selectable species of the analysis object. When the icon allocation button 62 is selected, the icon allocation screen is displayed on the display 3. FIG. 18 shows an example of an icon allocation screen. As shown in the drawing, the icon allocation screen 40 is, similar to the species name setting screen 36, provided with an apparatus status display area 41, paper feed button 42, main screen call button 55, and save button 65, as well as an icon allocation setting area 71. The icon allocation setting area 71 is provided with setting areas 71a through 71d corresponding to the species selection icons 45a through 45d, “Icon 1,” “Icon 2,” “Icon 3,” “Icon 4” are displayed horizontally to the setting areas 71a through 71d, suggesting the respectively corresponding icon. A region among the setting areas 71a through 71d is selected when the fingertip of a user touches the setting area corresponding to the icon for which the user wants to change the setting. For example, when the user wants to change the setting of the species selection icon 45d, the user selects the setting area 71d. When one setting area is selected in this way, the species selection dialogue is displayed. FIG. 19 shows a screen displaying the species selection dialogue. A plurality of species selection buttons 73a through 73c are displayed in the species selection dialogue 72. A page switching button 74 and close button 75 are also displayed in the species selection dialogue 72. The buttons 73a through 73c are identical to the species buttons 60e through 60g of the first species selection screen 34. That is, “Rabbit” is displayed on the button 73a, and “Other 2” is displayed on the button 73b, and “Other 3” is displayed on the button 73c. The species selection dialogue 72 has a plurality of pages, and when the page switching button 74 is selected, the controller 9 switches the display to another page. On the other pages are displayed similar buttons of other species on the first species selection screen 34 and second species selection screen. Therefore, When a desired species selection button is not displayed on the displayed page, the user touches the page switching button 74 with a fingertip and the display is switched to another page. When the close button 75 is selected, the dialogue 72 closes. When the user changes the species selection icon 45d, the user selects one button among the species selection buttons. In this case, the user selects the button 73a which displays “Rabbit.” Thus, the dialogue 72 closes. FIG. 20 shows another example of an icon allocation screen. The display of the icon allocation setting area 71d is changed to “Rabbit,” indicating that rabbit has been allocated to the species selection button 45d. To save this setting, the user selects the save button 65. Thus, the controller 9 stores the icon allocation setting in the memory 21.

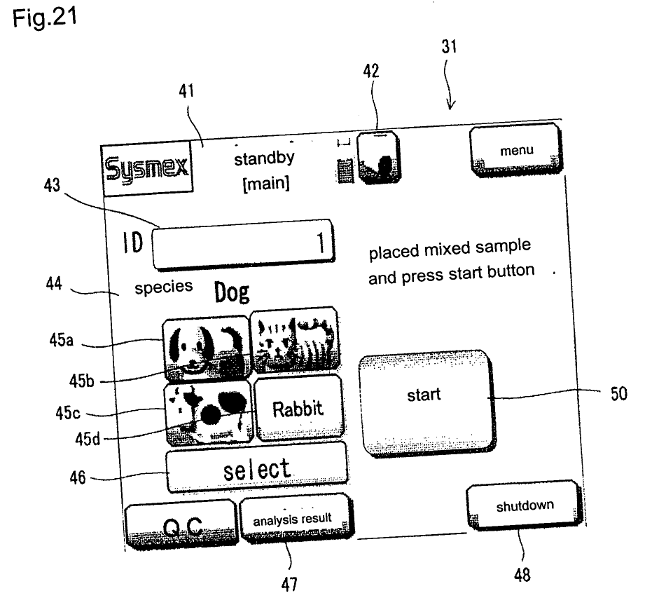

After the icon allocation setting has been changed in this way, the user touches the main screen call button 55 with a fingertip to call issue a call for the main screen 31. When the call for the main screen 31 is received, the controller 9 displays the main screen 31 on the display 3. Therefore, the icon allocation change process ends. FIG. 21 shows the main screen after the icon allocation change. After the icon allocation change, the display of the species selection icon 45d has been changed to “Rabbit” as shown in the drawing. Thus, the user can easily select rabbit as the measurement species. To stop the operation of the biological sample analyzer 1, the user touches the shutdown button 48 on the main screen 31 with a fingertip. When the selection of the shutdown button 48 has been received (step S29: YES), the controller 9 stops the biological sample analyzer 1. When the user wants to change another setting, the routine returns to the process fo step S22, and the processes subsequent to step S22 are repeated.

The biological sample analyzer 1 can not only set icons for the species set by the species selection icons 45a through 45d as described above, the analyzer can also mutually substitute the positions of the species selection icons 45a through 45d. For example, a user can display dog and cat buttons and set cat in the setting area 71a and set dog in the setting area 71b by selecting the page switching button 74. Thus, in the main screen 31 shown in FIG. 4, the species selection icon 45b (cat icon) is disposed at the position of the species selection icon 45a, and the species selection icon 45a (dog icon) is disposed at the position of the species selection icon 45b. Moreover, although the biological sample analyzer 1 of the present embodiment can move a species selection icon to any position among the species selection icons 45a through 45d, the present invention is not limited to this arrangement inasmuch as the biological sample analyzer 1 may also be configured to allow the species selection icon to be moved to any optional position on the main screen 31.

Although the biological sample analyzer 1 displays four types of species on the main screen 31 shown in FIG. 4, the present invention is not limited to this arrangement inasmuch as the biological sample analyzer 1 may also be configured to change the number of species displayed on the main screen. For example, when dog is set in all of the setting areas 71a through 71d in the screen 40 shown in FIG. 19, all the species selection icons 45a through 45d may be dog icons. Thus, a single species may be displayed on the main screen.

Furthermore, the biological sample analyzer 1 may be configured so as to allow a changeable number of species icons displayed on the main screen 31 shown in FIG. 4. For example, when the save button 65 is selected while the setting areas 71b through 71d are blank in the screen 40 shown in FIG. 19, only the species icon 45a is displayed on the main screen 31. In this case, the biological sample analyzer 1 may be configured so as to automatically change the size of the species icon according to the number of species icons displayed on the main screen 31. For example, when a single species icon is displayed on the main screen 31, a species icon having the size of the area for arranging the species selection icons 45a through 45d may be displayed in the allocated area; and in the case of two species icons, one species icon having the size of a first area for the disposition of the species selection icons 45a and 45b may be displayed in a first area, and a species icon having the size of a second area for the disposition of the species icon 45c and 45d may be displayed in a second area.

Furthermore, the biological sample analyzer 1 may also be configured so as to automatically substitute a selected species for any one of the species selection buttons 45a through 45d when the selector button 46 is selected on the main screen 31 and analysis is performed under the condition of the selected species, Moreover, an icon indicating the selected species may automatically be displayed on the main screen 31.

Although the biological sample analyzer 1 analyzes blood particles in the present embodiment, the present invention is not limited to this analysis inasmuch as, for example, the present invention is also applicable to apparatuses that perform blood coagulation measurements, immunological analysis and urine analysis.

Although the biological sample analyzer 1 of the present embodiment is capable of analyzing dog, cat, cow, horse, and rabbit, other species may be substituted, or pig, sheep, goat, deer, mouse and the like may also be analyzed as well.

Although the terms “icon” and “button” are used in the above description, “button” and “icon” mean the same thing. That is, “icon” is a term representing a higher concept “button.”

What is claimed is:

1. A sample analyzer for analyzing samples of a plurality of species of animals, comprising:
a display;
a memory for storing first icon data representing a first icon and second icon data representing a second icon, the first icon showing a first species of animal and being used for selecting a first analysis for a sample from the first species of an animal, and the second icon showing a second species of animal and being used for selecting a second analysis for a sample from the second species of an animal;
a screen display means for displaying a screen on the display, the screen comprising the first icon; and
a change receiving means for receiving a change of the first icon on the screen to the second icon.

2. The sample analyzer of claim 1,
wherein the memory further stores third icon data representing a third icon and fourth icon data representing a forth icon, the third icon showing a third species of animal and being used for selecting a third analysis for a sample from the third species of an animal, and the fourth icon showing a fourth species of animal and being used for selecting a fourth analysis for a sample from the fourth species of an animal;
wherein the screen further comprises the third icon; and
wherein the change receiving means further receives a second change of the third icon on the screen to the fourth icon.

3. The sample analyzer of claim 1,
wherein the screen is an analysis start screen for receiving start instructions for starting the first analysis and the second analysis.

4. The sample analyzer of claim 1,
wherein the analysis start screen is a main screen which is automatically displayed on the display after starting the sample analyzer.

5. The sample analyzer of claim 4,
wherein the first icon is automatically selected when the main screen is automatically displayed on the display.

6. The sample analyzer of claim 1,
wherein the memory further stores a first analysis condition for the first analysis and a second analysis condition for the second analysis; and
wherein the sample analyzer further comprises an analysis means for analyzing a sample of the first species of an animal under the first analysis condition when the first icon is selected, and analyzing a sample of the second species of an animal under the second analysis condition when the second icon is selected.

7. The sample analyzer of claim 6, further comprising
an analysis condition change receiving means for receiving changes of the first and the second analysis conditions.

8. The sample analyzer of claim 1,
wherein the memory further comprises selection icon data representing a selection icon and a fifth icon data representing a fifth icon, the fifth icon showing a fifth species of animal and being used for selecting a fifth analysis of a sample from the fifth species of an animal;
wherein the screen further comprises the selection icon; and
wherein the screen display means displays the fifth icon on the display when the selection icon is selected.

9. The sample analyzer of claim 1, further comprising
an icon data receiving means for receiving an input of at least a part of the second icon data.

10. The sample analyzer of claim 1, further comprising
a form changing means for changing a form of the first icon on the screen when the first icon is selected.

11. The sample analyzer of claim 1,
wherein the first icon comprises a graphic image representing the first species of animal.

12. The sample analyzer of claim 1,
wherein the first species of animal is selected from the group consisting of a dog, a cat, a cow, a horse, a pig, a sheep, a goat, a deer, a mouse, and a rabbit.

13. The sample analyzer of claim 1,
wherein a sample of the first species of an animal and a sample of the second species of an animal are blood.

14. A sample analyzer for analyzing samples of a plurality of species of animals, comprising:
a display;
a memory for storing first icon data representing a first icon and second icon data representing a second icon; the first icon showing a first species of animal and being used for selecting a first analysis of a sample from the first species of an animal, and the second icon showing a second species of animal and being used for selecting an analysis of a sample from the second species of an animal;
a screen display means for displaying a screen on the display, the screen comprising the first icon on a first position of the screen and the second icon on a second position of the screen; and
a change receiving means for receiving a change of a position of the first icon from the first position to the second position and a change of a position of the second icon from the second position to the first position.

15. The sample analyzer of claim 14,
wherein the memory further stores third icon data representing a third icon; the third icon showing a third species of animal and being used for selecting a third analysis of a sample from the third species of an animal;
wherein the screen further comprises the third icon on a third position of the screen; and
wherein the change receiving means further receives a change of the position of the first icon from the first or the second position to the third position and a change of a position of the third icon from the third position to the first or the second position.

16. The sample analyzer of claim 14,
wherein the memory further stores fourth icon data representing a fourth icon; the fourth icon showing a fourth species of animal and being used for selecting a fourth analysis of a sample from the fourth species of an animal; and
wherein the change receiving means receives a change of the first icon on the screen to the fourth icon.

17. A sample analyzer for analyzing samples of a plurality of species of animals, comprising:
a display;
a memory for storing first icon data representing a first icon and second icon data representing a second icon; the first icon showing a first species of animal and being used for selecting a first analysis of a sample from the first species of an animal, and the second icon showing a second species of animal and being used for selecting a second analysis of a sample from the second species of an animal;
a screen display means for displaying a screen on the display, the screen comprising at least one first icon; and
a change receiving means for receiving a change of a number of species of animals shown on the screen.

18. The sample analyzer of claim 17,
wherein the change receiving means changes the screen from a first screen which has one first icon to a second screen which has more than two first icons.

19. The sample analyzer of claim 17,
wherein the change receiving means changes a number of icons shown on the screen.

20. The sample analyzer of claim 17,
wherein the change receiving means changes a size of icon shown on the screen based on the number of icons shown on the screen.
